(19) United States
(12) Patent Application Publication
(10) Pub. No.: US 2010/0256323 A1
(43) Pub. Date: Oct. 7, 2010

(54) PREPOLYMERS AND POLYMERS FOR ELASTOMERS

(75) Inventors: Phillip S. Athey, Lake Jackson, TX (US); Woo-Sung Bae, Rosharon, TX (US)

Correspondence Address: The Dow Chemical Company, P.O. BOX 1967, Midland, MI, 48641, US

(73) Assignee: Dow Global Technologies Inc.

(21) Appl. No.: 12/675,874

(22) PCT Filed: Sep. 17, 2008
(86) PCT No.: PCT/US08/76585

§ 371 Date: Mar. 1, 2010

Related U.S. Application Data
(60) Provisional application No. 60/974,255, filed on Sep. 21, 2007.

(51) Int. Cl. C08G 18/10; C07C 265/00

(52) U.S. Cl. 528/85; 560/330

(57) ABSTRACT

The present invention is to the preparation and application of isocyanate based polymers, particularly polyurea elastomeric polymers. By incorporating polybutadiene with a natural oil based polyol in a thermoplastic system, particularly in the formation of a prepolymer the produced elastomer display good chemical resistance. Such prepolymers are an isocyanate terminate prepolymer wherein the polyol composition for the production of the prepolymer comprises a polybutadiene polyol and a natural oil based polyol.

### FIELD OF THE INVENTION

The present invention involves polyols, prepolymers, especially prepolymers of isocyanates and the polyols, preferably prepolymers useful for making elastomers made from the polyols, the prepolymers or combinations thereof.

### BACKGROUND OF THE INVENTION

Elastomers generally stretch under tension, have a high tensile strength and retract rapidly to the original dimension when the applied stress is released. Such elastomers can be used in a variety of applications including open casting techniques, injection molding and spray coating of surfaces.

Spray elastomers are a relatively young class of polyurethane elastomeric materials which have been introduced to the coatings industry about 20 years ago. Over the past decade, these spray applied polyurethane and polyurea polymers have found rapid acceptance in the protective coating industry due to their high reactivity, speed of application and mechanical strength and toughness. Such elastomers are widely used as coating on various substrates, such as metals, plastic, wood and concrete. For example, large containers, pipe housing, etc. are items which are subject to highly abrasive conditions and can be protected by an elastic, wear resistance covering.

Recently contractors and applicators have felt encouraged by their success to venture into harsher applications environments like chemical processing infrastructure, power generation, paper mills or mining.

However due to the severity of the chemical and/or thermal exposure spray elastomers have performed only marginally in these applications and have not been proven as a viable alternative to incumbent protection solutions like epoxy, polyester or vinylester coatings.

U.S. Pat. No. 6,797,789 describes a phenolic/polyurea elastomeric coating system reported to have improved chemical resistance. Such a system is based on an isocyanate quasi-prepolymer of an isocyanate and the other reactive component contains an amine-terminated polyether polyols, amine-terminated chain extenders and phenolic resins. U.S. Pat. No. 5,077,349 describes highly flexible polyurethane plastics and coatings which are resistant to chemicals and a process for their production. The reactive systems have a polyisocyanate component which is reacted with a hydroxy terminated polybutadiene polyol, water, alkaline earth metal hydroxides or oxides and organic auxiliaries like bitumen and additives. The polymer is processed with e.g. rollers or spatulas and is particularly suitable for large-area seals on concrete surfaces like e.g. garage decks or bridges.

While polybutadiene polyols gives elastomers with good chemical resistance, due to the expense of such polyols, it is desirable to find a substitutes which provide for a cost advantage while providing elastomers with good physical properties and good chemical resistance. It would also be desirable if a portion of the polyol could be produced from a renewable resource.

The objective of the present invention is to provide for non-cellular isocyanate based polymers which exhibit good chemical resistance, specifically acid resistance while preserving optimal set rates and flowability. The polymers also have good adhesion properties to allow attachment of the polymers to a substrate to provide a protective coating.

### SUMMARY OF THE INVENTION

It has now been found prepolymers prepared from at least one polyol derived from a renewable resource in combination with a polybutadiene polyol, when used in making elastomers, provides for the formation of elastomers having good physical and chemical resistance properties.

The present invention is to an isocyanate terminate prepolymer having an isocyanate (NCO) content of from 5 to 25 weight percent comprising the reaction product of a stoichiometric excess of one or more di- or polyisocyanates with a first polyol composition wherein the first polyol composition comprises from 10 to 90 weight percent of at least one natural oil based polyol; from 10 to 90 weight percent of a at least one polybutadiene polyol; and optionally in the presence of additional polyol or polyols.

In a further embodiment the polybutadiene polyol has a functionality of 1.8 to 2.1 and an average molecular weight of 500 to 10000.

In yet another embodiment, the natural oil based polyol is at least one polyester polyol or fatty acid derived polyol which is the reaction product of at least one initiator and a at least one fatty acid, a mixture of fatty acids or derivatives of fatty acids comprising at least about 45 weight percent monounsaturated fatty acids or derivatives thereof, wherein the polyol is derived from an initiator having an average of 1.7 to 4 reactive groups. In still a further embodiment, the natural oil based polyol has an average molecular weight of 500 to 5000.

In a further aspect of the invention, the invention is an elastomer comprising the mixing of

a) an isocyanate terminate prepolymer having an isocyanate (NCO) content of from 5 to 25 weight percent comprising the reaction product of a stoichiometric excess of one or more di- or polyisocyanates with a first polyol composition wherein the first polyol composition comprises from 10 to 90 weight percent of at least one natural oil based polyol; from 10 to 90 weight percent of at least one polybutadiene polyol; and optionally in the presence of additional polyol or polyols;

b) a second polyol composition wherein any polyol which is not a polybutadiene polyol or natural oil polyol is a polyol or polyol blend having a nominal functionality of 1.8 to 2.5 and an average molecular weight of 500 to 10,000;

c) optionally in the presence of chain extenders and/or cross linkers, and

d) optionally in the presence of catalysts and other additives known per se in the production of elastomers.

In another aspect, the invention is a process for producing an elastomer comprising admixing

a) an isocyanate terminate prepolymer having an isocyanate (NCO) content of from 5 to 25 weight percent comprising the reaction product of a stoichiometric excess of one or more di- or polyisocyanates with a first polyol composition wherein the first polyol composition comprises from 10 to 90 weight percent of at least one natural oil based polyol; from 10 to 90 weight percent of at least one polybutadiene polyol; and optionally in the presence of additional polyol or polyols;

b) a second polyol composition wherein any polyol which is not a polybutadiene polyol or natural oil polyol is a polyol or polyol blend having a nominal functionality of 1.8 to 2.5 and an average molecular weight of 500 to 10,000;

c) optionally in the presence of chain extenders and/or cross linkers, and

d) optionally in the presence of catalysts and other additives known per se in the production of elastomers.

In another aspect, the invention is an article, coating, adhesive, binding, or thermoplastic comprising the elastomer of the invention or formed from the prepolymer of or formed using the process of the invention.

### BRIEF DESCRIPTION OF THE DRAWINGS

FIG. 1 is a graph of the stress/strain curves for elastomers of comparative example 7 after exposure to sulfuric or nitric acid for various times. The elastomer of comparative example 7 is based on prepolymer having a natural oil based polyol Soy used in FIG. 1 represents the prepolymer based on polyol S used in the working examples.

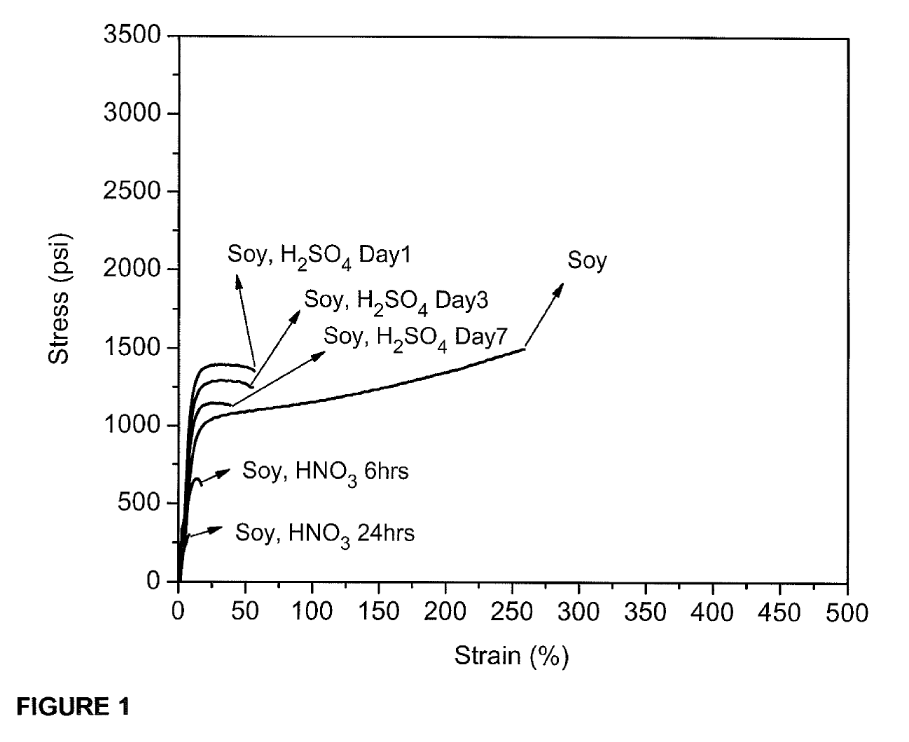

FIG. 2 is a graph of the stress/strain curves for elastomers of comparative example 8 after exposure to sulfuric or nitric acid for various times. The elastomer of comparative example 8 is based on prepolymer having a polyether polyol. Voranol used in FIG. 2 represents the prepolymer based on polyol V used in the working examples.

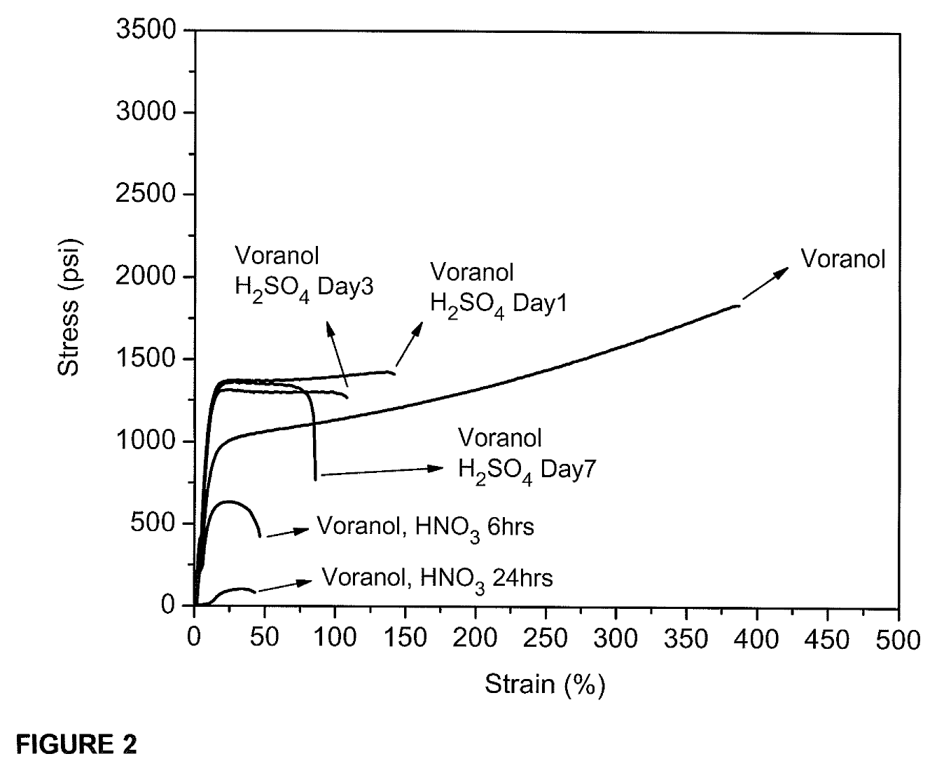

FIG. 3 is a graph of the stress/strain curves for elastomers of comparative example 11 after exposure to sulfuric or nitric acid for various times. The elastomer of comparative example 11 is based on prepolymer having polyether and polybutadiene polyols.

FIG. 4 is a graph of the stress/strain curves for elastomers of example 4 after exposure to sulfuric or nitric acid for various times. The elastomer of example 4 is based on prepolymer having a natural oil based to polybutadiene polyol weight ratio of 1:3.

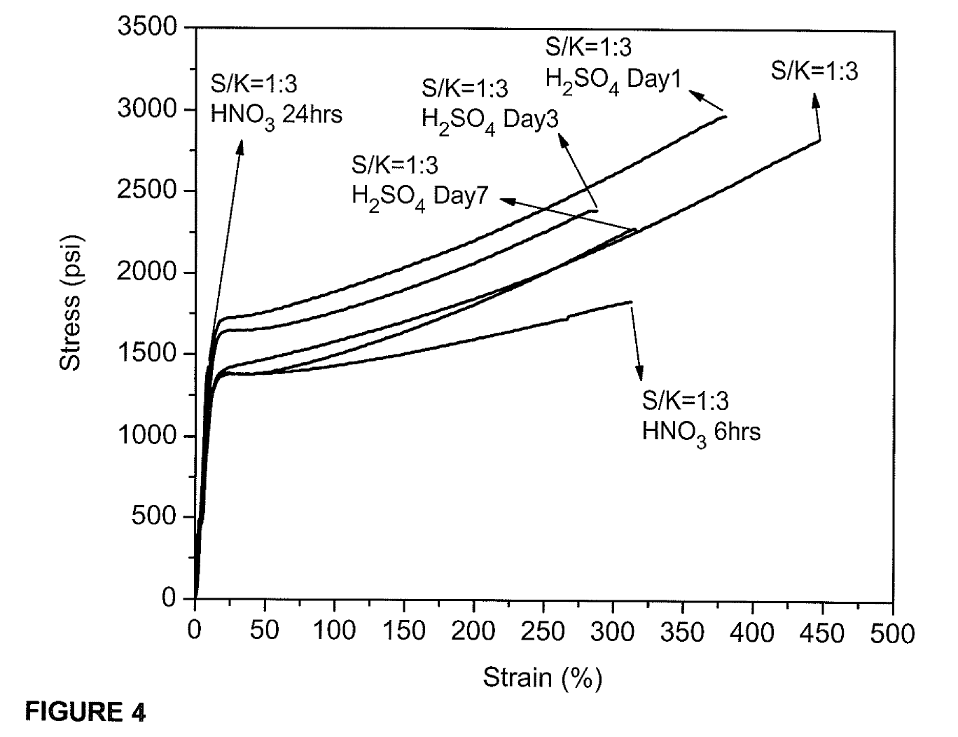

FIG. 5 is a graph of the stress/strain curves for elastomers of example 5 after exposure to sulfuric or nitric acid for various times. The elastomer of example 5 is based on prepolymer having a natural oil based to polybutadiene polyol weight ratio of 1:1.

FIG. 6 is a graph of the stress/strain curves for elastomers of example 6 after exposure to sulfuric or nitric acid for various times. The elastomer of example 6 is based on prepolymer having a natural oil based to polybutadiene polyol weight ratio of 3:1.

### DETAILED DESCRIPTION

The present invention relates to the preparation and application of plural component coating systems that exhibit comparable or enhanced chemical resistance as compared to systems based on a polybutadiene or polyether based elastomer. The improved properties make such coating systems suitable for use in corrosive environments.

As used herein the term polyols are materials having at least one group containing an active hydrogen atom capable of undergoing reaction with an isocyanate. Preferred among such compounds are materials having at least two hydroxyls, primary or secondary, or at least two amines, primary or secondary, carboxylic acid, or thiol groups per molecule. Compounds having at least two hydroxyl groups per molecule are especially preferred due to their desirable reactivity with polyisocyanates.

As used herein the term “conventional polyol” or “additional polyol” is used to designate a polyol other than a polybutadiene polyol or a natural oil polyol.

The term “natural oil based polyol” (NOBP) is used herein to refer to compounds having hydroxyl groups which compounds are isolated from, derived from or manufactured from natural oils, including animal and vegetable oils, preferably vegetable oils.

The term “fatty acid derived polyol” is used herein to refer to NOBP compounds which are derived from fatty acids available from natural oils. For instance, fatty acids are reacted with compounds ranging from air or oxygen to organic compounds including amines and alcohols. Frequently, unsaturation in the fatty acid is converted to hydroxyl groups or to a group which can subsequently be reacted with a compound that has hydroxyl groups such that a polyol is obtained.

The presence of polybutadiene and NOBP as part of the polyol component for producing the non-cellular polymers, i.e. elastomers, of the present invention, give polymers with good chemical resistance. It is believed the hydrophobic nature of the hydroxy terminated polybutadiene and NOBP resin imparts chemical resistance to such elastomers against various media like aqueous acids and bases, some solvents and aqueous solutions of various salts.

When the polyol component b), chain extenders, and/or cross-linkers contain active amine hydrogen groups, the reaction of such active amine hydrogen groups with the isocyanate component of a) results in the formation or urea linkages. When the polyol component b), chain extenders, and/or cross-linkers contain active hydroxyl hydrogen groups, the reaction of such active hydroxyl hydrogen groups with the isocyanate component of a) results in the formation or polyurethane linkages. Thus the elastomers of the present invention may be a polyurethane, polyurea or a polyurethane/polyurea hybrid elastomers.

The polybutadiene used in the present invention is a non-branched hydroxyl-terminated polybutadiene which contains an average of 1.8 to 2.0 terminal hydroxyl groups and have a weight average molecular weight of 500 to 10,000, preferably from 700 to 8,000 and more preferably about 1,000 to 5,000. More preferably the polybutadiene has a weight average molecular weight of 1,500 to 4,000. Such non-branched polybutadienes are derived from anionic polymerization and are available commercial, for example, from Sartomer as Krasol™ LBH 2000, 3000 and 5000.

Preferably the polybutadiene component is used as a portion of the first polyol composition used in producing a prepolymer. Generally the polybutadiene will be from 10 to 90 weight percent of the first polyol composition. Preferably the polybutadiene will comprise at least 20 and more preferably at least 35 weight percent of the first polyol composition. In one embodiment, the first polyol comprises at least 45 weight percent of polybutadiene. The polybutadiene may comprise up to 75 and more preferably up to 66 weight percent of the first polyol. In one embodiment the first polyol comprises up to 55 weight percent of polybutadiene.

Natural oil based polyol (NOBP) are polyols based on or derived from renewable resources such as natural and/or genetically modified (GMO) plant vegetable seed oils and/or animal source fats and/or algae. Such oils and/or fats are generally comprised of triglycerides, that is, fatty acids linked together with glycerol. Preferred are vegetable oils that have at least about 50 percent and more preferably at least 80 weight percent unsaturated fatty acids in the triglyceride. Even more preferred are natural products which contains at least about 85 percent by weight unsaturated fatty acids. In one embodiment, the natural oil contains 90 weight percent or more of unsaturated fatty acids.

Examples of vegetable and animal oils that may be used include, but are not limited to, soybean oil, safflower oil, linseed oil, castor oil, corn oil, sunflower oil, olive oil, canola oil, sesame oil, cottonseed oil, palm oil, rapeseed oil, tung oil, fish oil, or a blend of any of these oils. Alternatively, any partially hydrogenated or epoxidized natural oil or genetically modified natural oil can be used to obtain the desired hydroxyl content. Examples of such oils include, but are not limited to, high oleic safflower oil, high oleic soybean oil, high oleic peanut oil, high oleic sunflower oil (such as NuSun sunflower oil), high oleic canola oil, and high erucic rapeseed oil (such as Crumbe oil). Natural oil polyols are well within the knowledge of those skilled in the art, for instance as disclosed in Colvin et al., UTECH Asia, Low Cost Polyols from Natural Oils, Paper 36, 1995 and “Renewable raw materials—an important basis for urethane chemistry:” Urethane Technology: vol. 14, No. 2, April/May 1997, Crain Communications 1997, WO 01/04225, WO 040/96882; WO 040/96883; U.S. Pat. No. 6,686,435, U.S. Pat. No. 6,433,121, U.S. Pat. No. 4,508,853, U.S. Pat. No. 6,107,403, US publications 20060041157, and 20040242910.

Examples of preferred vegetable oils include, for example, those from castor, soybean, olive, peanut, rapeseed, corn, sesame, cotton, canola, safflower, linseed, palm, sunflower seed oils, or a combination thereof. Examples of animal products include lard, beef tallow, fish oils and mixtures thereof. A combination of vegetable and animal based oils/fats may also be used. Preferably NOBP are derived from soybean and/or castor and/or canola oils.

Generally it is desirable to modify the natural materials to give the material isocyanate reactive groups or to increase the number of isocyanate reactive groups on the material. Preferably such reactive groups are a hydroxyl group. Several chemistries can be used to prepare the polyols. Such modifications of a renewable resource include, for example, epoxidation, as described in U.S. Pat. No. 6,107,433 or in U.S. Pat. No. 6,121,398; hydroxylation, such as described in WO 2003/029182; esterification such as described in U.S. Pat. No. 6,897,283; 6,962,636 or 6,979,477; hydroformylation as described in WO 2004/096744; grafting such as described in U.S. Pat. No. 4,640,801; or alkoxylation as described in U.S. Pat. No. 4,534,907 or in WO 2004/020497. The above cited references for modifying the natural products are incorporated herein by reference.

In another embodiment, NOBP are obtained by a combination of the above modification techniques as disclosed in PCT Publications WO 2004/096882 and 2004/096883, and Applicant's copending application Ser. No. 60/676,348 entitled “Polyester Polyols Containing Secondary alcohol Groups and Their Use in Making Polyurethanes Such as Flexible Polyurethane Foams”, the disclosures of which are incorporated herein by reference.

In an even more preferred embodiment, polyols of the present invention are produced by the transesterification of vegetable oil based monomers (VOB's) as described in WO2004/096882 with a hydroxyl or polyhydroxyl functional species. As described therein, these VOB's are characterized by a structure containing from 0 to 3 primary OH species on a fatty acid moiety. The functionality distribution of these VOB's can be controlled and varied based on the starting composition of the fatty acids or by separation of VOB's themselves or their precursors. In brief, the process involves a multi-step process wherein the animal or vegetable oils/fats is subjected to transesterification and the constituent fatty acids recovered. This step is followed by hydroformylating carbon-carbon double bonds in the constituent fatty acids to form hydroxymethyl groups, and then forming a polyester or polyether/polyester by reaction of the hydroxymethylated fatty acid with an appropriate initiator compound. Preferably, the NOBP used in the present invention are polyester polyols based on the reaction of a hydroxymethylated fatty acid with an initiator.

The initiator for use in the multi-step process for the production of polyol are as those generally used in the production of conventional polyether polyols.

Preferably the initiator is selected from the group consisting of neopentylglycol; 1,2-propylene glycol; trimethylolpropane; pentaerythritol; sorbitol; sucrose; glycerol; diethanolamine; alkanediols such as 1,6-hexanediol, 1,4-butanediol; 1,4-cyclohexane diol; 2,5-hexanediol; ethylene glycol; diethylene glycol, triethylene glycol; bis-3-aminopropyl methylamine; ethylene diamine; diethylene triamine; 9(1)-hydroxymethyloctadecanol, 1, 3 or 1,4-bishydroxymethylcyclohexane or a mixture thereof; 8,8-bis(hydroxymethyl)tricyclo[5,2,1,0^{2,6}]decene; Dimerol alcohol; hydrogenated bisphenol; 9,9(10,10)-bishydroxymethyloctadecanol; 1,2,6-hexanetriol and combination thereof.

Suitable inititors are also include the above noted initiators which are alkoxlyated with ethylene oxide or a mixture of ethylene and at least one other alkylene oxide to give an alkoxylated initiator with a molecular weight of 200 to 6000, especially from 400 to 2000. Preferably the alkoxylated initiator has a molecular weight from 500 to 1000.

The fatty acid derived polyol advantageously has an average number of functional groups reactive with aliphatic or aromatic isocyanate groups, preferably hydroxyl groups, per molecule of at least about 1.7, preferably at least about 1.8, more preferably at least about 1.9, most preferably at least about 1.95, and preferably at most about 3.5, more preferably at most about 3, and in one embodiment most preferably at most about 2. In one embodiment, the fatty acid derived polyol advantageously has at least about 45, preferably at least about 65, more preferably at least about 80, most preferably at least about 85 and up to 100 percent by weight molecules having 2 groups reactive with aromatic isocyanate groups, preferably hydroxyl groups.

Furthermore, the fatty acid derived polyol advantageously has an number average molecular weight at least sufficient to form elastomers, that is advantageously at least about 1000, preferably at least about 1500, more preferably at least about 2000, and preferably at most about 5000, more preferably at most about 4000, most preferably at most about 3000.

Preferably the NOBP is used as a portion of the first polyol composition used in producing a prepolymer. Generally the NOBP will be from 10 to 90 weight percent of the first polyol composition. Preferably the NOPB will comprise at least 20 and more preferably at least 35 weight percent of the first polyol composition. In one embodiment, the first polyol comprises at least 45 weight percent of NOBP. Preferably the NOBP will comprise up to 75 and more preferably up to 66 weight percent of the first polyol. In one embodiment the first polyol comprises up to 55 weight percent of NOBP.

For the second polyol compostion b), polyols known in the art for producing polyurethane or polyurea elastomers can be used. Representative polyols include polyether polyols, polyester polyols, polyhydroxy-terminated acetal resins, and hydroxyl-terminated amines Examples of these and other suitable isocyanate-reactive materials are described more fully in U.S. Pat. No. 4,394,491. Alternative polyols that may be used include polyalkylene carbonate-based polyols and polyphosphate-based polyols. Preferred are polyether or polyester polyols. More preferred are polyether polyols prepared by adding an alkylene oxide, such as ethylene oxide, propylene oxide, butylene oxide or a combination thereof, to an initiator having from 2 to 8, preferably 2 to 6 and more preferable from 2 to 4 active hydrogen atoms. Catalysis for this polymerization can be either anionic or cationic, with catalysts such as KOH, CsOH, boron trifluoride, or a double cyanide complex (DMC) catalyst such as zinc hexacyanocobaltate or quaternary phosphazenium compound.

A blend of polyols may be used and such a blend will generally have an average functionality of 1.8 to 4 and more preferably from 1.8 to 3, more preferably from 1.8 to 2.5. For the production of polyurethane elastomer, the functionality of the polyol blend is from 1.8 to 2.2. The average functionality of the polyol blend does not include any chain extenders or cross-linkers described more fully herein. The average equivalent weight of the polyol or polyol blend is generally from 500 to 3,000, preferably from 750 to 2,500 and more preferably from 1,000 to 2,200.

Exemplary initiators for polyether polyols include, for example, ethanediol, 1,2- and 1,3-propanediol, diethylene glycol, dipropylene glycol, tripropyleneglycol; polyethyleneglycol, polypropylene glycol; 1,4-butanediol, 1,6-hexanediol, glycerol, pentaerythritol, sorbitol, sucrose, neopentylglycol; 1,2-propylene glycol; trimethylolpropane glycerol; 1,6-hexanediol; 2,5-hexanediol; 1,4-butanediol; 1,4-cyclohexane diol; ethylene glycol; diethylene glycol; triethylene glycol; 9(1)-hydroxymethyloctadecanol, 1,3- or 1,4-bishydroxymethylcyclohexane or a mixture thereof; 8,8-bis(hydroxymethyl)tricyclo[5,2,1,0^{2,6}]decene; Dimerol alcohol (36 carbon diol available from Henkel Corporation); hydrogenated bisphenol; 9,9(10,10)-bishydroxymethyloctadecanol; 1,2,6-hexanetriol; and combination thereof.

Other initiators for polyether polyols include linear and cyclic compounds containing an amine. Exemplary polyamine initiators include ethylene diamine, neopentyldiamine, 1,6-diaminohexane; bisaminomethyltricyclodecane; bisaminocyclohexane; diethylene triamine; bis-3-aminopropyl methylamine; triethylene tetramine various isomers of toluene diamine; diphenylmethane diamine; N-methyl-1,2-ethanediamine, N-Methyl-1,3-propanediamine, N,N-dimethyl-1,3-diaminopropane, N,N-dimethylethanolamine, 3,3′-diamino-N-methyldipropylamine, N,N-dimethyldipropylenetriamine, aminopropyl-imidazole.

Illustrative polyester polyols may be prepared from organic dicarboxylic acids having from 2 to 12 carbon atoms, preferably aromatic dicarboxylic acids having from 8 to 12 carbon atoms, and polyhydric alcohols, preferably diols, having from 2 to 12, preferably from 2 to 8 and more preferably 2 to 6 carbon atoms. Examples of dicarboxylic acids are succinic acid, glutaric acid, adipic acid, suberic acid, azelaic acid, sebacic acid, decanedicarboxylic acid, malonic acid, pimelic acid, 2-methyl-1,6-hexanoic acid, dodecanedioic acid, maleic acid and fumaric acid. Preferred aromatic dicarboxylic acids are phthalic acid, isophthalic acid, terephthalic acid and isomers of naphthalene-dicarboxylic acids. Such acids may be used individually or as mixtures. Examples of dihydric and polyhydric alcohols include ethanediol, diethylene glycol, triethylene glycol, 1,2- and 1,3-propanediol, dipropylene glycol, 1,4-butanediol and other butanediols, 1,5-pentanediol and other pentanediols, 1,6-hexanediol, 1,10-decanediol, glycerol, and trimethylolpropane. Illustrative of the polyester polyols are poly(hexanediol adipate), poly(butylene glycol adipate), poly(ethylene glycol adipate), poly(diethylene glycol adipate), poly(hexanediol oxalate), poly(ethylene glycol sebecate), and the like.

While the polyester polyols can be prepared from substantially pure reactants materials, more complex ingredients can be used, such as the side-stream, waste or scrap residues from the manufacture of phtalic acid, terephtalic acid, dimethyl terephtalate, polyethylene terephtalate and the like. Other source is the recycled PET (polyethelene terephtalate). After transesterification or esterification the reaction products can optionally be reacted with an alkylene oxide.

Another class of polyesters which may be used are polylactone polyols. Such polyols are prepared by the reaction of a lactone monomer; illustrative of which is δ-valerolactone, ε-caprolactone, ε-methyl-ε-caprolactone, ξ-enantholactone, and the like; with an initiator that has active hydrogen-containing groups; illustrative of which is ethylene glycol, diethylene glycol, propanediols, 1,4-butanediol, 1,6-hexanediol, trimethylolpropane, and the like. The production of such polyols is known in the art; see, for example, U.S. Pat. Nos. 3,169,945, 3,248,417, 3,021,309 and 3,021,317. The preferred lactone polyols are the di-, tri-, and tetra-hydroxyl functional ε-caprolactone polyols known as polycaprolactone polyols.

For the production of polyureas, component b) or c) will contain amine terminated molecules. When part of the second polyol b), such active amine hydrogen containing materials are preferably amine-terminated polyethers. Such amine-terminated polyols have a molecular weight of greater than 1,000 and generally greater than 1,500. The preferred amine-terminated polyethers should be selected from aminated diols or triols, and a blend of aminated diols and/or triols may be used. In particular, primary and secondary amine-terminated polyethers with a molecular weight greater than 1000, even more desirably greater than 1500, a functionality from about 2 to about 6, and an amine equivalent weight of from about 750 to about 4000 are preferred. In one embodiment, such amine-terminated polyethers having a functionality of from about 2 to about 3 are used. These materials may be made by various methods known in the art.

The amine-terminated polyethers may be, for example, polyether resins made from an appropriate initiator to which lower alkylene oxides, such as ethylene oxide, propylene oxide, butylene oxide, or mixtures thereof, are added with the resulting hydroxyl-terminated polyol then being aminated. When two or more oxides are used, they may be present as random mixtures or as blocks of one or the other polyether. In the amination step, it is highly desirable that the terminal hydroxyl groups in the polyol be essentially all secondary hydroxyl groups for ease of amination. The polyols so prepared are then reductively aminated by known techniques, such as described in U.S. Pat. No. 3,654,370, for example, the contents of which are incorporated herein by reference. Normally, the amination step does not completely replace all of the hydroxyl groups. However, the greatest majority of hydroxyl groups are replaced by amine groups. Therefore, the amine-terminated polyether resins generally have greater than about 90 percent of their active hydrogens in the form of amine hydrogens.

Examples of such amine terminated polyethers are JEFFAMINE® brand series of polyether amines available from Huntsman Corporation. They include JEFFAMINE® D-2000, JEFFAMINE® D-4000, JEFFAMINE® T-3000 and JEFFAMINE® T-5000. Other similar polyether amines are commercially available from BASF and Arch Chemicals.

The isocyanate-terminated prepolymer for use in the present inventions are prepared by standard procedures well known to a person skilled in the art and such as disclosed in U.S. Pat. Nos. 4,294,951; 4,555,562; 4,182,825 or PCT Publication WO2004074343. The components are typically mixed together and heated to promote reaction of the polyols and the polyisocyanate. The reaction temperature will commonly be within the range of about 30° C. to about 150° C.; a more preferred range being from about 60° C. to about 100° C. The reaction is advantageously performed in a moisture-free atmosphere. An inert gas such as nitrogen, argon or the like can be used to blanket the reaction mixture. If desired, an inert solvent can be used during preparation of the prepolymer, although none is needed. A catalyst to promote the formation of urethane bonds may also be used.

The isocyanate is used in stoichiometric excess and reacted with the polyol component using conventional prepolymer reaction techniques to prepare prepolymers having from 5 to 25 weight percent free NCO groups. The prepolymers generally have from 8 to 20 weight percent free NCO groups, preferably from 10 to 18 weight percent, and more preferably from 14 to 17 weight percent.

As the prepolymer contains a polybutadiene and NOBP based polyol, separate prepolymers may be produced, one based on the isocyanate and polybutadiene and the second based on isocyanate and NOBP. The resulting prepolymers can then be blended together. Alternatively, the prepolymer may be prepared by reacting the polybutadiene and NOBP polyol with the isocyanate simultaneously in a one-pot procedure.

Suitable polyisocyanates for producing the prepolymers include aromatic, cycloaliphatic and aliphatic isocyanates. Such isocyanates are well known in the art.

Examples of suitable aromatic isocyanates include the 4,4′-, 2,4′ and 2,2′-isomers of diphenylmethane diisocyante (MDI), blends thereof and polymeric and monomeric MDI blends, toluene-2,4- and 2,6-diisocyante (TDI) m- and p-phenylenediisocyanate, chlorophenylene-2,4-diisocyanate, diphenylene-4,4′-diisocyanate, 4,4′-diisocyanate-3,3′-dimethyldiphenyl, 3-methyldiphenyl-methane-4,4′-diisocyanate and diphenyletherdiisocyanate and 2,4,6-triisocyanatotoluene and 2,4,4′-triisocyanatodiphenylether.

A crude polyisocyanate may also be used in the practice of this invention, such as crude toluene diisocyanate obtained by the phosgenation of a mixture of toluene diamine or the crude diphenylmethane diisocyanate obtained by the phosgenation of crude methylene diphenylamine. In one embodiment, TDI/MDI blends are used.

Examples of aliphatic polyisocyanates include ethylene diisocyanate, 1,6-hexamethylene diisocyanate, 1,3- and/or 1,4-bis(isocyanatomethyl)cyclohexane (including cis- or trans-isomers of either), isophorone diisocyanate (IPDI), tetramethylene-1,4-diisocyanate, methylene bis(cyclohexaneisocyanate) (H₁₂MDI), cyclohexane 1,4-diisocyanate, 4,4′-dicyclohexylmethane diisocyanate, saturated analogues of the above mentioned aromatic isocyanates and mixtures thereof.

Derivatives of any of the foregoing polyisocyanate groups that contain biuret, urea, carbodiimide, allophonate and/or isocyanurate groups can also be used. These derivatives often have increased isocyanate functionalities and are desirably used when a more highly crosslinked product is desired.

Preferably the polyisocyanate is diphenylmethane-4,4′-diisocyanate, diphenylmethane-2,4′-diisocyanate, polymers or derivatives thereof or a mixture thereof. In one preferred embodiment, the isocyanate-terminated prepolymers are prepared with 4,4′-MDI, or other MDI blends containing a substantial portion or the 4.4′-isomer or MDI modified as described above. Preferably the MDI contains 45 to 95 percent by weight of the 4,4′-isomer.

It is also possible to use one or more chain extenders for the production of polyurethane polymers and elastomers of the present invention. The presence of a chain extending agent provides for desirable physical properties, of the resulting polymer. The chain extenders may be blended with the polyol component ii) or may be present as a separate stream during the formation of the polyurethane polymer. For purposes of this invention, a chain extender is a material having two isocyanate-reactive groups per molecule and an equivalent weight per isocyanate-reactive group of less than 400, preferably less than 300 and especially from 31-125 daltons. Representative of suitable chain-extending agents include polyhydric alcohols, aliphatic diamines including polyoxyalkylenediamines, aromatic diamines and mixtures thereof. The isocyanate reactive groups are preferably hydroxyl, primary aliphatic or aromatic amine or secondary aliphatic or aromatic amine groups. Representative chain extenders include ethylene glycol, diethylene glycol, 1,3-propane diol, 1,3- or 1,4-butanediol, dipropylene glycol, 1,2- and 2,3-butylene glycol, 1,6-hexanediol, neopentylglycol, tripropylene glycol, ethylene diamine, 1,4-butylenediamine, 1,6-hexamethylenediamine, phenylene diamine, 1,5-pentanediol, 1, 3 or 1,4-bishydroxymethylcyclohexane or mixtures thereof, 1,6-hexanediol, bis(3-chloro-4-aminophenyl)methane, 3,3′-dichloro-4,4-diaminodiphenylmethane, 4,4′-diaminodiphenylmethane, bisphenol-A; bisphenol-F, 1,3-propane di-p-aminobenzene, methylene bisorthochloroaniline (MOCA), 1,3-cyclohexandiol, 1,4-cyclohexanediol; 2,4-diamino-3,5-diethyl toluene 1,3-cyclohexane dimethanol, 1,4-cyclohexane dimethanol, and mixtures thereof. If used, chain extenders are typically present in an amount from about 0.5 to about 20, especially about 2 to about 16 parts by weight per 100 parts by weight of the polyol component. Such chain extenders are generally added in the production of elastomer. Chain extenders are generally added to the second polyol component, however; if desired, the chain extenders added to the isocyanate terminated prepolymer to partially react out the free isocyanate groups.

Crosslinkers may also be included in formulations for the production of polyurethane polymers of the present invention. For purposes of this invention “crosslinkers” are materials having three or more isocyanate-reactive groups per molecule and an equivalent weight per isocyanate-reactive group of less than 400. Crosslinkers preferably contain from 3-8, especially from 3-4 hydroxyl, primary amine or secondary amine groups per molecule and have an equivalent weight of from 30 to about 200, especially from 50-125. Examples of suitable crosslinkers include diethanol amine, monoethanol amine, triethanol amine, mono- di- or tri(isopropanol) amine, glycerine, trimethylol propane, pentaerythritol, sorbitol, diethyltoluenediamine (DETA), meta-dianiline, and other diamine cross-linkers known to those skilled in the art.

For producing a polyurethane based elastomer, amounts of crosslinkers generally used are from about 0.1 to about 1 part by weight, especially from about 0.25 to about 0.5 parts by weight, per 100 parts by weight of polyols.

When producing polyurea elastomers, the urea content may come from reaction of the isocyanate with amine terminated polyols present in the second polyol b) or provided by the presence of amine terminated chain extenders or amine terminated prepolymers. Thus polyurea elastomers referred to herein are those formed from reaction mixtures having at least about 50 percent of the active hydrogen groups in the form of amine groups. Preferably, the reaction mixtures have at least about 60 percent and more preferably about 70 percent active amine hydrogen groups in the form of amine groups. In a more preferred embodiment, the reaction mixtures have at least about 90 percent of the active hydrogen groups in the form of amine groups.

The ratio of equivalents of isocyanate groups in the polyisocyanate a) to the active hydrogens in polyol component b) plus those present for any added chain extenders or crosslinkers is generally from 85 to 115. Preferably the isocyanate index is at a ratio of 90 to 110 and more preferably from 95 to 110. The isocyanate index is known to those skilled in the art and is the mole equivalents of isocyanate (NCO) divided by the total mole equivalents of isocyanate-reactive hydrogen atoms present in a formulation, multiplied by 100.

To obtain adequate curing rates, a catalyst may be included within the polyol component. Suitable catalysts include the tertiary amine and organometallic compounds such as described in U.S. Pat. No. 4,495,081. When using an amine catalyst advantageously it is present in from 0.1 to 3, preferably from 0.1 to 1 and more preferably from 0.4 to 0.8 weight percent by total weight of polyol and optional chain extending agent. When the catalyst is an organometallic catalyst, advantageously it is present in from 0.001 to 0.2, preferably from 0.002 to 0.1 and more preferably from 0.01 to 0.05 weight percent by total weight of polyol and optional chain extending agent. Particularly useful catalysts include in the case of amine catalysts; triethylenediamine, bis(N,N-dimethylaminoethyl)ether and di(N,N-dimethylaminoethyl)amine and in the case of the organometallic catalysts; stannous octoate, dibutyltin dilaurate, and dibutyltin diacetate. Combinations of amine and organometallic catalysts may be employed.

The viscosity of the prepolymers may be reduced by the mixing with diluents known to those skilled in the art. One preferred diluent is propylene carbonate.

Various other additives generally known to those skilled in the art can be added to the elastomers. For example, pigments, such as titanium dioxide and/or carbon black, may be incorporated in the elastomer system to impart color properties. Pigments may be in the form of solids or the solids may be pre-dispersed in a resin carrier. Reinforcements, for example, flake or milled glass, and fumed silica, may also be incorporated in the elastomer system to impart certain properties. Other additives such as UV stabilizers, antioxidants, air release agents, adhesion promoters, or structural reinforcing agents may be added to the mixture depending on the desired characteristics of the end product. These are generally known to those skilled in the art. The amount of any such additive is not taken into consideration when determining the weight percent of polybutadiene in the final polymer.

The elastomers of the present invention are applicable for use in applications which require heavy duty anticorrosion properties like floors in chemical or food processing plants, pickup-bed linings, reservoirs linings, storage tanks, floors etc. Alternatively the polymers can be used for applications requiring higher thermal resistance or applications requiring high hydrolysis resistance like marine coatings.

The polyurethane polymer prepared according to the process of this invention is a solid or a microcellular polyurethane polymer. Such a polymer is typically prepared by intimately mixing the reaction components at room temperature or a slightly elevated temperature for a short period and then pouring the resulting mixture into an open mold, or injecting the resulting mixture into closed mold, which in either case is heated. The mixture on reacting out takes the shape of the mold to produce a polyurethane polymer of a predefined structure, which can then when sufficiently cured be removed from the mold with a minimum risk of incurring deformation greater than that permitted for its intended end application. Suitable conditions for promoting the curing of the polymer include a mold temperature of typically from 20° C. to 150° C., preferably from 35° C. to 75° C., and more preferably from 45° C. to 55° C. Such temperatures generally permit the sufficiently cured polymer to be removed from the mold typically in from 1 to 10 minutes and more typically from 1 to 5 minutes after intimately mixing the reactants. Optimum cure conditions will depend on the particular components including catalysts and quantities used in preparing the polymer and also the size and shape of the article manufactured.

For elastomeric spray coatings, the components are generally applied via processing through plural high pressure spray machines. The plural component equipment combines the two components a) and b) while the b) component generally includes other additives as described above. The isocyanate a) and polyol b) are preferably combined or mixed under high pressure. In a preferred embodiment, they are impingement mixed directly in the high-pressure spray equipment. This equipment for example includes: GUSMER H-2000, GUSMER H-3500, GUSMER H-20/35 and Glas-Craft MH type proportioning units fitted with either a GUSMER GX-7, GUSMER GX-7 400 series or GUSMER GX-8 impingement mix spray gun. The two components are mixed under high pressure inside the spray gun thus forming the coating/lining system, which is then applied to the desired substrate via the spray gun. The use of plural component spray equipment, however, is not critical to the present invention and is included only as one example of a suitable method for mixing the isocyanate and polyol components of the present invention.

The following examples are provided to illustrate the present invention. The examples are not intended to limit the scope of the present invention and should not be so interpreted. All percentages are by weight unless otherwise noted.

A description of the raw materials used in the examples is as follows.
K is the designation used for polybutadiene polyol (diol) with a trade designation of Krasol LBH2000 having a reported functionality of about 1.9 and average MW 2000; (range 1800-2500), OH# 40-65 mg KOH/g, viscosity @ 25° C. 5000-20000 mPa s. Krasol is a trademark of Sartomer Europe.
DETA is diethyl toluene diamine (DETDA) obtained from Albemarle, which is a difunctional aromatic amine chain extender.
T-5000 is a 5000 MW polyetheramine available from The Hanson Group, LLC. A reported synonym for T-5000 is alpha, alpha′, alpha″-1,2,3-propanetriyltris(omega-(2-aminomethylethoxy)-poly-oxy(methyl-1,2-ethanediyl)).
Polylink 4200 is a secondary aromatic diamine having a molecular weight of 310 and an apparent hydroxyl number of 362, available from The Hanson Group LLC.
D-2000 is a plolyetheramine having a molecular weight of 2,000 available from The Hanson Group LLC. A reported synonym for T2000 is alpha-(2-aminomethylethyl)-omega-(2-aminomethylethoxy)-poly(oxy(methyl-1,2-ethanediyl)).
ISO-1 is ISONATE* 50-OP, a monomeric MDI; having a 2,4′/4,4′ isomer ratio of about 50/50 available from The Dow Chemical Company.
V is the designation VORANOL* V220-110 a 1000 MW all PO diol, available from The Dow Chemical Company.
   *ISONATE and VORANOL are trademarks of The Dow Chemical Company
S is the designation for a natural oil based polyol (NOBP). This (2000 MW diol) polyol (2000 MW diol) is based on the polymerization of methyl hydroxymethylstearate (HMS). The polyol is produced by the reaction of mixed 1,3 and 1,4-cyclohexanedimethanol commercially available from The Dow Chemical Company under the trade designation Unoxol™ (36.2 g) and methyl 9-hydroxymethylstearate (165.0 g; >90% purity). The reactants are charged to a 500 ml 3-neck round bottom flask, equipped with a short path condenser, receiving flask, nitrogen inlet, nitrogen outlet to mineral oil bubbler, and magnetic stirrer. The mixture is heated to 120° C. in an oil bath moderated by thermocouple controller, and kept under nitrogen atmosphere while mixing. At 120° C., the contents are degassed and backfilled with nitrogen 3 times, then catalyst (dibutyltin oxide) is added at 1000 ppm based on charge weight. The temperature is increased by 10° C. per 30 minutes until 190° C. is reached.

This temperature and conditions are held until the methanol stops coming over (usually over 4 hours). The same product is obtained by continuing the conditions overnight. At the conclusion of visible methanol evolution, the temperature is maintained for a minimum 2 hour hold. Then vacuum is applied to remove traces of methanol, drive molecular weight to the targeted level or a combination thereof. It is desirable to remove as much methanol as possible using vacuum and temperatures above 120° C.

At the conclusion of the high vacuum step (at less than 0.5 mm) the material is then cooled to 25° C. and transferred to a glass jar. The resulting viscosity, molecular weight, and hydroxyl number are evaluated and found to be 2380 cps (22° C./Spindle #34), 824 Mn, and hydroxyl number of 136.

### Plaque Formation

Plaques are prepared using a modified procedure described by W. R. Schmeal, Polymeric Engineering and Science, 119, 13, page 1173. Rather than the use of a drawdown bar, the prepolymer and polyol component are mixed through a static mixer, passed through a plastic lined dual roller press. After being prepared, the plaques are laid flat and allowed to sit for 15 to 20 min before the plastic lining is removed. The resulting plaques are further cured overnight at ambient conditions before any mechanical tests are performed.

Acid Resistance Test: Acid resistant tests are done according to the procedures of test method ASTM D543-06. For the HNO₃ resistance test, three different lengths of immersion time (6 hrs, 24 hrs, and 7 days) at room temperature are employed, and 5 replicas of samples are utilized for each exposure time. The dog-bones shaped coupons made from the polyurea plaques are weighed and placed in 60 ml glass containers which are filled with HNO₃ aqueous solution (40%). After various immersion time, the dog-bones are rinsed with deionized H₂O, blotted dry and reweighed. The dog-bones are dried under ambient condition for 24 hrs and weighed again. The same experimental procedures are utilized for H₂SO₄ resistance test, except samples are immersed in H₂SO₄ aqueous solution (30%) at 70° C. for seven days, with a dog bone coupon removed each day.

ASTM D 1708 is used to measure tensile strength and data is applied to generation of stress-strain curves.
ASTM D412 is used to measure percent elongation and data is applied to generation of stress-strain curves.
The yield point is where the curve bends (inflection point) for the generated stress-strain curves.
ASTM D624 to measure tear strength.
Thermal properties of plaques were evaluated using thermo gravimetric analysis (TGA) which was conducted on TGA Q50 (TA Instruments) for selected samples ranging from 25° C. to 500° C. with 10° C./min in air.

Prepolymer preparation: The prepolymers are prepared in batches by first adding the isocyanate to a glass jar while purging with nitrogen followed by addition of the polyol components. The mixtures are stirred 5 minutes at 500 rpm under nitrogen purging and then placed in a preset 70° C. oven for three hours. The targeted free NCO content is approximately 15.7 wt percent of the prepolymer. The prepolymers are prepared using the polyols as given in Table 1, where the ratio of polyols is based on a weight ratio. Examples labeled C1 to C6 are controls. Approximately 1 gram of benzoyl chloride is added to 300 grams of prepolymer.

TABLE 1
Prepolymer Component

| Prepolymer | Polyol | % NCO |
|---|---|---|
| C1 | S | 15.54 |
| C2 | V | 14.55 |
| C3 | K | 15.67 |
| C4 | K:V | 15.41 |
| | ratio 1:3 | |
| C5 | K:V | 15.71 |
| | ratio 1:1 | |
| C6 | K:V | 16.32 |
| | ratio 3:1 | |
| Example 1 | S:K | 15.59 |
| | Ratio 1:3 | |
| Example 2 | S:K | 15.58 |
| | Ratio 1:1 | |
| Example 3 | S:K | 15.73 |
| | Ratio 3:1 | |

For the formation of elastomers, the above prepolymers are mixed with the following polyol composition: 22 wt % DETA; 10 wt % T-5000; 10 wt % Polylink 4200 and 58 wt % D-2000. The formulations used for the production of plaques are given in Table 2. Examples labeled C are comparatives.

TABLE 2
Formulations for Producing Plaques

| | | Isocyanate side | | |
|---|---|---|---|---|
| | Prepolymer | Prepolymers | Propylene Carbonate | Polyol |
| Example | Example | (g) | (g) | (g) |
| C7 | C1 | 190.34 | 11.5 | 183 |
| C8 | C2 | 214.55 | 7.2 | 196.8 |
| C9 | C3 | 197.9 | 10.5 | 188.47 |
| C10 | C4 | 173.6 | 8.2 | 161.0 |
| C11 | C5 | 190.3 | 9.5 | 181.2 |
| C12 | C6 | 165.3 | 10.0 | 162.7 |
| 4 | 1 | 179.9 | 10.5 | 168.9 |
| 5 | 2 | 179.7 | 10.7 | 168.8 |
| 6 | 3 | 172.8 | 10.2 | 163.8 |

Propylene carbonate is added in the above formulations to provide for an even volume of prepolymer/polyol for ease of producing the plaques and also acts to reduce the viscosity of the prepolymer.

The mechanical property of the plaques resulting from the comparative formulations C7 to C12 and examples 4 to 6 are given in table 3.

TABLE 3
Tensile strength, Yield point, and % Elongation collected from
plaque samples.

| | Tensile | Yield | |
|---|---|---|---|
| | Strength | Point | % |
| Example | (psi) | (psi) | Elongation |
| C7 | 1500 | 1020 | 260 |
| C8 | 1800 | 960 | 380 |
| C9 | 2150 | 1300 | 310 |
| C10 | 2080 | 1150 | 413 |
| C11 | 1800 | 1200 | 340 |
| C12 | 2270 | 1200 | 427 |
| E4 | 2820 | 1320 | 446 |
| E5 | 2350 | 1300 | 358 |
| E6 | 2260 | 1480 | 302 |

The data indicates plaques generated from hybrid polyols based on NOBP and polybutadiene polyol (PBD) exhibit a better percent elongation than the homopolyol values, indicating the hybrid polyol have a synergistic effect on properties of the fabricated plaques. This results in the formation of polyurea plaques with enhanced tensile strength. The yield stress is not generally improved by using such hybrid polyol system, however; the inclusion of a NOBP in the formulation does have a substantial adverse affect on the yield stress.

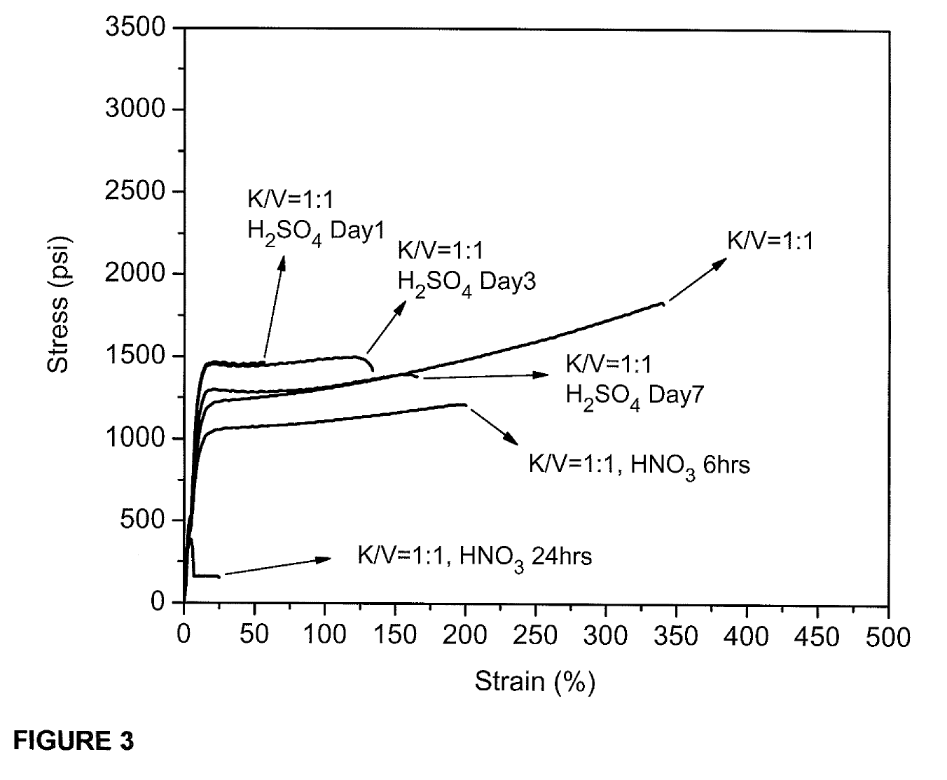

The mechanical properties of the acid treated plaques are measured to verify their performance as a function of exposure time to acid. As shown in FIG. 3 (comparative example 5) the stress-strain curve collected from K/V (1:1) hybrid polyurea plaques illustrate the 30% H₂SO₄ solution at 70° C. was not a severe condition for K/V hybrid, however; the condition of the 40% HNO3 exposure at room temperature dramatically degrades the mechanical properties after 24 hrs. Additionally, the K/V hybrid plaques seemed to maintain a high level of ductility after acid exposure, an indication that the PBD component not only enhanced the mechanical properties, but also the acid durability of the K/V plaques. Overall, a change in the yield strength was observed after H₂SO₄ treatment, which was probably attributed to the generation of organic sulfate. The formation of organic sulfates could tend to boost the ionic intermolecular interaction among the soft segment domains, which may translate into slightly higher modulus and yield points.

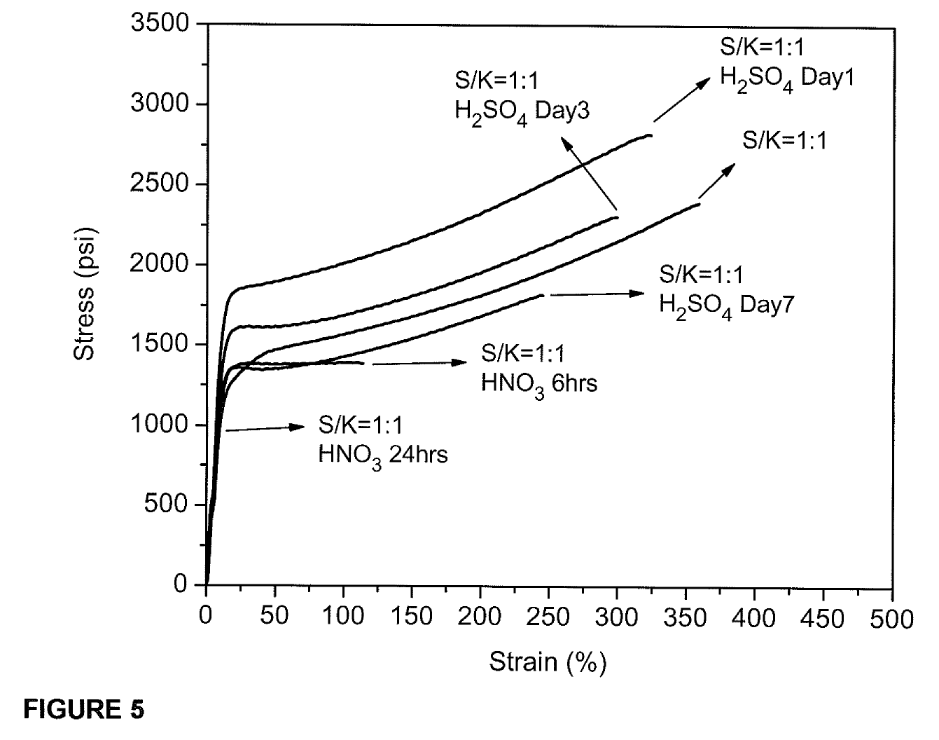

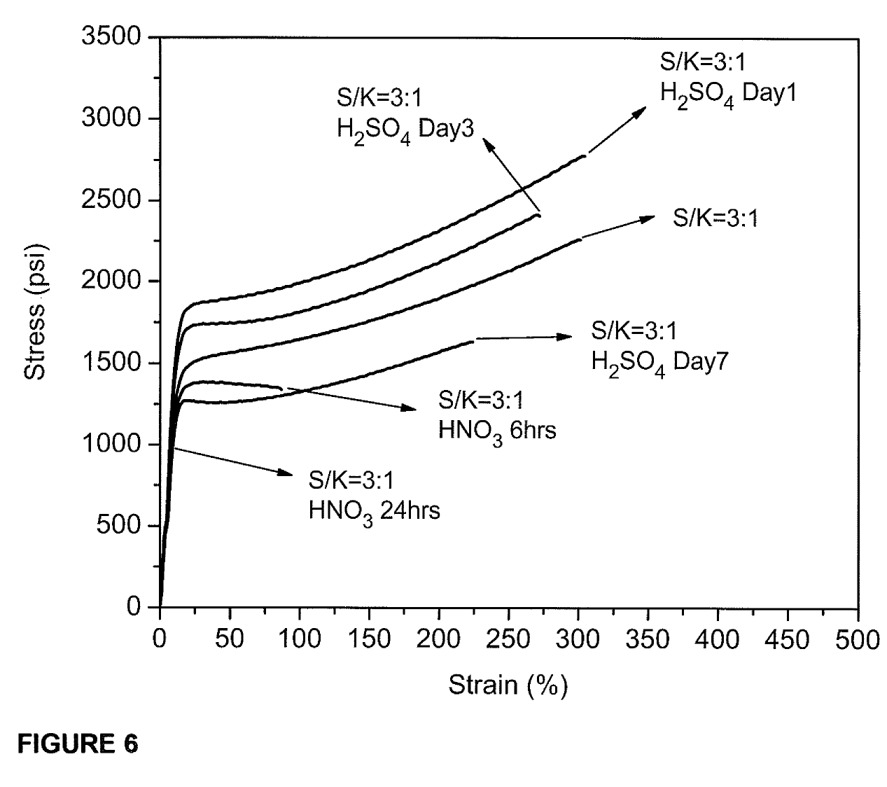

The Stress-Strain curves collected from S/K 1:3 plaques as a function of acid exposure time (FIG. 6) reveals a similar degradation with the exception being that the % elongation of sulfuric acid treated sample was close to that of the original plaque. Furthermore, the SK=1:3 maintains acceptable mechanical properties even after 6 hrs of nitric acid exposure, as depicted by the yield point. FIGS. 5 and 6 illustrate the mechanical property variations as a function of the acid exposure time for S/K=1:3 and 1:1 hybrid polyurea plaques indicating that the ductility and acid resistance of the plaques are weakened as the concentration of NOBP is increased. Plaques, with the S/K=1:3 formulation demonstrate the best post acid mechanical properties out of the polyol blends evaluated

In addition to measuring the acid resistant abilities of the hybrid polyurea plaques, the thermal degradation profile of the plaques is evaluated to determine their potential applications in thermally aggressive environments. Therefore, thermal gravimetric analysis (TGA) is employed for measuring degradation temperatures of plaque samples throughout acid resistance test. Table 4 gives the TGA data collected from plaques as a function of the acid exposure time. Two major degradation temperatures are detected at around 280 and 390° C. for all of the samples. It is theorized the polyproplylene oxide (PPO) group of the polyol component degrades at 280° C. with this temperature decreasing linearly upon acid exposure, probably due to molecular scissoring. The polybutadiene backbone appears to undergo degradation at approximately 390° C. and has a consistent degradation temperature regardless of acid exposure time. It should be noted that the utilization of more polybutadiene component results in plaques losing less weight after 300° C. (data not shown).

TABLE 4

| Materials | TGA |
|---|---|
| S | 277 |
| H₂SO₄ 1 day | 276, 352, |
| day 4 | 272, 354 |
| day7 | 270, 351 |
| HNO₃ 6 h | 259, 349 |
| 24 hr | 257, 345 |
| V | 281 |
| H₂SO₄ 1 day | 277 |
| day 4 | 265 |
| day7 | 261 |
| HNO₃ 6 h | 264 |
| 24 hr | 252 |
| K | |
| H₂SO₄ 1 day | 281 |
| day 4 | 276 |
| day7 | 274 |
| HNO₃ 6 h | 263 |
| 24 hr | 260 |
| K/V 1:1 | 282, 392 |
| H₂SO₄ 1 day | 273, 387 |
| day 4 | 272, 386 |
| day7 | 271, 391 |
| HNO₃ 6 h | 268, 391 |
| 24 hr | 258, 396 |
| K/V 1:3 | Not measured |
| H₂SO₄ 1 day | |
| day 4 | |
| day7 | |
| HNO₃ 6 h | |
| 24 hr | |
| K/V 3:1 | Not measured |
| H₂SO₄ 1 day | |
| day 4 | |
| day7 | |
| HNO₃ 6 h | |
| 24 hr | |
| K/S 1:3 | 300, 403 |
| H₂SO₄ 1 day | 291, 397 |
| day 4 | 275, 392 |
| day7 | 275, 384 |
| HNO₃ 6 h | 266, 402 |
| 24 hr | 265, 403 |
| K/S 1:1 | 291, 398 |
| H₂SO₄ 1 day | 282, 392 |
| day 4 | 272, 391 |
| day7 | 271, 394 |
| HNO₃ 6 h | 266, 400 |
| 24 hr | 264, 414 |
| K/S 3:1 | 277 |
| H₂SO₄ 1 day | 272, 351 |
| day 4 | 260, 344 |
| day7 | 257, 336 |
| HNO₃ 6 h | 254, 345 |
| 24 hr | 255, 342 |

The TGA data obtained from S/K hybrid plaques demonstrate a similar thermal degradation as the of K/V hybrid plaques. The data indicates 290° C. represent the degradation temperature (Td) of the PPO and NOBP polyol backbone, while the polybutadiene of PBD degrades at about 400° C. As a higher concentration of PBD is blended into the plaques, an improvement in the thermal stability of the plaques is noted. However, regardless of the S/K ratio, the first degradation temperature is reduced as a function of exposure time to acid.

Utilizing the natural oil based polyols with polybutadiene in polyurea spray evaluations, gives a similar pattern of properties and acid resistance as observed for the plaque formation.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. An isocyanate terminate prepolymer having an isocyanate (NCO) content of from 5 to 25 weight percent comprising the reaction product of a stoichiometric excess of one or more di- or polyisocyanates with a first polyol composition wherein the first polyol composition comprises from 10 to 90 weight percent of at least one natural oil based polyol; from 10 to 90 weight percent of at least one polybutadiene polyol; and optionally in the presence of additional polyol or polyols.

2. The prepolymer of claim 1 wherein the polybutadiene has functionality of 1.8 to 2.1 and an average molecular weight of 500 to 10,000.

3. The prepolymer of claim 1 or 2 wherein the polybutadiene has a molecular weight of 1,000 to 5,000.

4. The prepolymer of claim 3 wherein the polybutadiene comprises from 20 to 75 weight percent of the first polyol composition.

5. The prepolymer of claim 4 wherein the polybutadiene comprises from 35 to 66 weight percent of the first polyol composition.

6. The prepolymer of any one of the preceding claims wherein the natural oil based polyol is at least one polyester polyol or fatty acid derived polyol which is the reaction product of at least one initiator and at least one fatty acid or at least one derivative of a fatty acid.

7. The prepolymer of claim 6 wherein the initiator has an average of 1.7 to 4 reactive groups.

8. The prepolymer of claim 7 wherein the natural oil based polyol has an average molecular weight of 500 to 5,000.

9. The prepolymer of claim 6 wherein the natural oil based polyol comprises from 35 to 66 weight percent of the first polyol composition.

10. An elastomer comprising the mixing of
a) an isocyanate terminate prepolymer having an isocyanate (NCO) content of from 5 to 25 weight percent comprising the reaction product of a stoichiometric excess of one or more di- or polyisocyanates with a first polyol composition wherein the first polyol composition comprises from 10 to 90 weight percent of at least one natural oil based polyol; from 10 to 90 weight percent of at least one polybutadiene polyol; and optionally in the presence of additional polyol or polyols;
b) a second polyol composition wherein any polyol which are not a polybutadiene polyol or natural oil polyol which is not polybutadiene is a polyol or polyol blend having a nominal functionality of 1.8 to 2.5 and an average molecular weight of 500 to 10,000;
c) optionally in the presence of chain extenders and/or cross linkers, and
d) optionally in the presence of catalysts and other additives known per se in the production of elastomers.

11. A process for producing an elastomer comprising admixing
a) an isocyanate terminate prepolymer having an isocyanate (NCO) content of from 5 to 25 weight percent comprising the reaction product of a stoichiometric excess of one or more di- or polyisocyanates with a first polyol composition wherein the first polyol composition comprises from 10 to 90 weight percent of at least one natural oil based polyol; from 10 to 90 weight percent of at least polybutadiene polyol; and optionally in the presence of additional polyol or polyols;
b) a second polyol composition wherein any polyol which are not a polybutadiene polyol or natural oil polyol which is not polybutadiene is a polyol or polyol blend having a nominal functionality of 1.8 to 2.5 and an average molecular weight of 500 to 10,000;
c) optionally in the presence of chain extenders and/or cross linkers, and
d) optionally in the presence of catalysts and other additives known per se in the production of elastomers.
